(19) United States
(12) Patent Application Publication
(10) Pub. No.: US 2026/0026953 A1
(43) Pub. Date: Jan. 29, 2026

(54) BALLOON DELIVERY SYSTEM FOR THE IMPLANT DEPLOYMENT

(71) Applicant: Northwestern University, Evanston, IL (US)

(72) Inventors: Yonghui DING, Evanston, IL (US); Henry Oliver Tenadooah WARE, Evanston, IL (US); Cheng SUN, Evanston, IL (US); Guillermo Antonio AMEER, Evanston, IL (US)

(21) Appl. No.: 18/997,759

(22) PCT Filed: Jul. 21, 2023
(86) PCT No.: PCT/US2023/070743

§ 371 Date: Jan. 22, 2025

Related U.S. Application Data
(60) Provisional application No. 63/391,493, filed on Jul. 22, 2022.

(51) Int. Cl. A61F 2/966; A61F 2/958

(52) U.S. Cl. CPC A61F 2/966; A61F 2002/9583; A61F 2210/0004; A61F 2250/0067

(57) ABSTRACT

Provided herein are balloon delivery systems (BDSs) for deploying expandable implants at treatment locations within a subject. In particular provided herein are BDSs providing optimal deployment of bioresorbable vascular scaffolds (BVS). for example. within a coronary artery.

### CROSS-REFERENCE TO RELATED APPLICATIONS

This application claims the benefit of U.S. Provisional Patent Application No. 63/391,493, filed on Jul. 22, 2022, which is incorporated by reference herein.

### STATEMENT REGARDING FEDERAL FUNDING

This invention was made with government support under grant number HL141933 awarded by the National Institutes of Health. The government has certain rights in the invention.

### FIELD

Provided herein are balloon delivery systems (BDSs) for deploying expandable implants at treatment locations within a subject. In particular provided herein are BDSs providing optimal deployment of bioresorbable vascular scaffolds (BVS), for example, within a coronary artery.

### BACKGROUND

Percutaneous coronary intervention (PCI) has been widely used for the treatment of patients with coronary artery disease. However, PCI is often associated with stent sizing difficulties, such as stent under-expansion or stent malapposition (SM), which is an important predictor of restenosis, target vessel revascularization, and subacute thrombosis. Stent deployment systems play critical roles in determination of final stent dimensions and apposition.

### SUMMARY

Provided herein are balloon delivery systems (BDSs) for deploying expandable implants at treatment locations within a subject. In particular provided herein are BDSs providing optimal deployment of bioresorbable vascular scaffolds (BVS), for example, within a coronary artery.

In some embodiments, provided herein are catheter systems for placement of an implant within a subject, the system comprising: (a) a catheter comprising having distal and proximal ends; (b) an implant for insertion within a subject, wherein the implant comprises a lumen, wherein the catheter extends longitudinally through the lumen of the implant, and wherein the implant is capable of adopting a compacted conformation and an expanded conformation; (c) a stopper located at a position along the catheter that is proximal to the location of the implant on the catheter, wherein the stopper has a greater diameter than compacted conformation of the implant; and (d) a sheath that extends longitudinally around the catheter and envelops the implant; wherein the stopper prevents movement of the implant toward the proximal end of the catheter when the sheath is retracted toward the proximal end of the catheter. In some embodiments, the catheter comprises a shaft and an inflatable balloon located at or near a distal end of the shaft. In some embodiments, all or a portion of the balloon resides within the lumen of the implant. In some embodiments, the balloon is capable of adopting deflated and inflated states. In some embodiments, the deflated balloon can reside within the implant in the compacted conformation, but the inflated balloon is too large to reside within the implant in the compacted conformation. In some embodiments, a partially-inflated balloon can reside within the implant in the compacted conformation, but a fully-inflated inflated balloon is too large to reside within the implant in the compacted conformation. In some embodiments, catheter extends longitudinally through the stopper. In some embodiments, the stopper is attached to the catheter and resides at a fixed position on the catheter. In some embodiments, the sheath envelops the catheter, stopper, implant and balloon. In some embodiments, a partially-inflated balloon exerts force on the inside of the sheath, thereby holding the implant and the sheath in place with respect to the catheter. In some embodiments, the friction force between the sheath, the implant, and the stents are designed to bold them in place with respect to each other. In some embodiments, in case of the implant exhibiting self-expansion in nature and failing to facilitate sufficient friction force, then partially-inflated balloon can be used to exert force. In some embodiments, the sheath can be retracted toward the proximal end of the catheter over the catheter, stopper, implant and deflated balloon. In some embodiments, the sheath can be retracted to expose the stopper, implant and balloon. In some embodiments, the implant is a stent. In some embodiments, the stent is a coronary stent intended for placement in a coronary artery. In some embodiments, the stent is a bioresorbable vascular scaffold (BVS). In some embodiments, the implant is self-expanding or partially self-expanding. In some embodiments, the sheath prevents the implant from adopting an expanded conformation by holding the sheath in the compacted conformation. In some embodiments, the sheath prevents the implant from adopting an expanded conformation by preventing the sheath from contacting the physiologic environment outside the sheath. In some embodiments, retracting the sheath allows the implant to adopt the expanded conformation and/or to partially expand. In some embodiments, retracting the sheath and inflating the balloon forces the implant to adopt the expanded conformation. In some embodiments, the implant is a drug-eluting implant.

In some embodiments, provided herein are methods of placing an implant within a treatment site in a subject, the method comprising: (a) inserting the system herein into a subject such that the implant is aligned with the treatment site; (b) retracting the sheath toward the proximal end of the catheter, thereby exposing the implant to the treatment site; and (c) allowing the implant to self-expand and/or applying mechanical outward force on the implant to place the implant into the expanded conformation within the treatment site.

In some embodiments, provided herein are methods of placing an implant within a treatment site in a subject, the method comprising: (a) inserting the system described herein into a subject such that the implant is aligned with the treatment site; (b) retracting the sheath toward the proximal end of the catheter, thereby exposing the implant to the treatment site; and (c) inflating the balloon to apply mechanical outward force on the implant to expand the implant into the expanded conformation within the treatment site. In some embodiments, the balloon is in a non-inflated or partially-inflated state when the system is inserted into the subject and the implant is aligned with the treatment site. In some embodiments, methods further comprise a step after step (a) of deflating the balloon. In some embodiments, the balloon is deflated before step (c). In some embodiments, the balloon is deflated before step (b). In some embodiments, methods further comprise retracting the catheter from the treatment site.

### BRIEF DESCRIPTION OF THE DRAWINGS

FIG. 1. Schematic (top) and photograph (bottom) of a novel balloon delivery system (BDS) for optimal deployment of a self-expanding bioresorbable vascular scaffold (BVS).

FIG. 2. The self-expanding BVS deployment procedure by using an exemplary prepared BDS.

FIG. 3. Image of BVS device insertion into the transparent section distal to the hemostatic valve of the guiding catheter.

FIG. 4. Image depicting alignment marks on the sheath and balloon catheter near the proximal end (outside the subject) of the balloon catheter.

FIG. 5. Image depicting retraction of the sheath along the catheter.

FIG. 6A-D. An exemplary BVS delivery system enables the deployment of BVS into the coronary artery of swine models. The fabricated BVS (A) was crimped (B), assembled with the customized BVS delivery system (C), and implanted into the coronary artery of a pig model. (C-i) The schematic illustration of BVS deployment steps, including step 1 (S1), the assembled BVS on balloon catheter; S2, the retraction of the sheath with BVS stopped by the stopper; S3, the release of the BVS from the sheath; S4, the inflation of the balloon along with the expansion of the BVS; S5, the deflation of the balloon; and S6, the retraction of the balloon catheter. (C-ii) The photo of the crimped BVS locked inside a sheath. (C-iii) The photo of the assembled BVS with customized delivery system. (C-iv) the photo of expanded BVS on a balloon catheter. (D-i)

X-ray image from the angiography reveals left anterior descending coronary artery (LAD) and left circumflex artery (LCX) of a pig. (D-ii) X-ray image shows the inflated balloon to expand the BVS in LCX. (D-iii) The intravascular ultrasound (IVUS) image shows BVS struts (as indicated by arrows) following implantation.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “device” is a reference to one or more devices and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “biostable” refers to compositions or materials that do not readily break-down or degrade in a physiological or similar aqueous environment. Conversely, the term “biodegradeable” refers herein to compositions or materials that readily decompose (e.g., depolymerize, hydrolyze, are enzymatically degraded, disassociate, etc.) in a physiological or other environment.

### Detailed Description

Provided herein are balloon delivery systems (BDSs) for deploying expandable implants at treatment locations within a subject. In particular provided herein are BDSs providing optimal deployment of bioresorbable vascular scaffolds (BVS), for example, within a coronary artery.

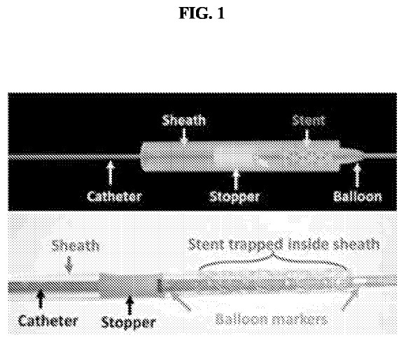

In some embodiments, systems comprising a sheath to restrain the implant (e.g., the BVS) and prevent its repositioning and/or self-expansion prior to the deployment in the target site (FIG. 1). In some embodiments, stopper within the deployment system prevents the implant (e.g., the BVS) from retracting during placement steps (e.g., while retracting the sheath) and to prevent the implant (e.g., the BVS) from moving away from the balloon.

In some embodiments, provided herein are systems (e.g., devices, components, etc.) and methods for placement of implantable devices within a subject. In some embodiments, the implantable devices comprising an overall tubular shape but may vary in physical shape. Implants that may be placed with the systems and methods herein include stents, such as vascular stents, esophageal stents, intestinal stents, biliary stents, ureteral stents, etc. However, the systems and methods herein may be used for placement of other non-stent implantable devices as well.

In some embodiments, an implant for use with the systems or methods herein is capable if adopting both compressed and expanded conformations. A tubular implant (e.g., stent) typically comprises a lumen extending from a proximal end of the implant to a distal end of the implant. In such embodiments, in the expanded conformation the cross-sectional dimensions of the lumen is greater than in the compressed conformation. In some embodiments, the lumen of the implant is fully expanded or substantially expanded (e.g., >95% full expansion, >90% pull expansion, >80% full expansion, >70% full expansion, etc.), while in the compressed conformation, the lumen of the implant is closed or nearly closed (e.g., <1% full expansion, <5% full expansion, <10% full expansion, <20% full expansion, etc.). In some embodiments, a catheter of a system herein is capable of passing through the lumen of an implant in the expanded or compressed conformation. In some embodiments, a balloon of a system herein, when in its deflated conformation, is capable of passing through the lumen of an implant the implant in the expanded conformation but not the compressed conformation. In other embodiments, a deflated balloon of a system herein is capable of passing through the lumen of an implant the implant in the expanded and compressed conformations. In some embodiments, a fully-inflated balloon of a system herein (e.g., inflated to the degree necessary to place the implant in its expanded conformation) is not capable of passing through the lumen of the implant in the expanded or compressed conformations. In some embodiments, balloon of a system herein, when in a partially-inflated conformation (e.g., for initial insertion of the system within the sheath), is not capable of passing through the lumen of the implant in the compressed conformation. In some embodiments, an implant is capable of residing within the lumen of a sheath of the systems herein when the implant is in a compressed conformation but not an expanded conformation.

In some embodiments, an implant is self-expanding (e.g., capable of transitioning from the compressed conformation to the expanded conformation without mechanical expansion). In some embodiments, an implant is partially self-expanding (e.g., capable of transitioning from the compressed conformation to a partially-expanded conformation without mechanical expansion but requiring mechanical expansion to reach a fully expanded conformation). In some embodiments, a self-expanding and/or partially self-expanding implant does not self-expand within the interior of a sheath of the systems herein (e.g., due to mechanical compression within the sheath, due to protection from the exterior (e.g., physiological) environment (e.g., liquid), etc.). In some embodiments, an implant is not self-expanding (e.g., does not transition from the compressed conformation to an expanded or partially-expanded conformation without mechanical expansion).

In some embodiments, the implant is made of any suitable material. In some embodiments, the stent is sterile, biocompatible, non-toxic, etc. In some embodiments, an implant is biodegradable (e.g., half-life of 1 week, 2 weeks, 1 month, 2 months, 6 months, 1 year, of more). In some embodiments, an implant is non-biodegradable. In some embodiments, an implant is placed permanently within a subject. In some embodiments, an implant is removable.

In some embodiments, an implant for use with the systems and methods herein is a stent. Stents may bind use in the vascular, digestive, biliary, urinary, or other body systems. In some embodiments, stents to be placed with the system and methods herein are vascular stents, such as those to be deployed in an artery of a subject. In particular embodiments, a stent is intended for placement at a treatment location within a coronary artery of a subject. In some embodiments, the stents herein find use, for example, in relieving pathologic strictures or obstructions in lumen (e.g., ureter, bile ducts, pancreatic ducts, etc.) of a subject, and are placed at the appropriate treatment locations. In some embodiments, a stent allows proper fluid flow through a lumen of a subject. In some embodiments, a stent finds use in treating or preventing occlusions, strictures, and/or leaks in a lumen of a subject. Embodiments herein are not limited by the type of stent, the locations of placement, or the indication that the stenting is intended to treat/prevent.

In some embodiments, the body of the stent comprises a thin and flexible material that allows the stent to approximate the inner wall of a lumen it is placed within. In some embodiments, the body of the stent is sufficiently resistant to compression to allow it to prevent and/or correct a stricture or narrowing in the lumen it is placed within.

In some embodiments, the stent body comprises a flexible material, such as silicone, a plastic (e.g., polyurethane, polyethylene, or blends thereof), Teflon, or other polymers/elastomers. Among natural and synthetic biodegradable polymers, chitosan, poly(4-hydroxybutyrate) (PHB), poly(ε-caprolactone) (PCL), poly (L-lactide) (PLLA) and poly(D,L-lactide) (PDLLA) and its copolymers or composites have been extensively investigated for use in resorbable devices.

In some embodiments, an implant (e.g., stent) for use with a placement system of method herein comprises a polymeric component. In some embodiments, a polymeric component comprises a polymer selected from a polyester, poly(diol citrate) (e.g., poly(butanediol citrate), poly(hexanediol citrate), poly(octanediol citrate), poly(decanediol citrate), poly(dodecanediol citrate), poly(hexadecanediol citrate), etc.), poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(D,L-lactide), poly(caprolactone), poly(trimethylene carbonate), polyester amide, or co-polymers or composites thereof.

In some embodiments, a polymeric component of an implant (e.g., stent) comprises a citric acid-based polymer. In some embodiments, a polymer is the polyesterification product of one or more acids (e.g., succinic acid, glutaric acid, adipic acid, pimelic acid, suberic acid, azelaic acid, sebacic acid, dodecanedioic acid, shorter or longer linear aliphatic diacids, citric acid, isocitric acid, aconitic acid, propane-1,2,3-tricarboxylic acid, trimesic acid, itaconic acid, maleic acid, etc.) and one or more diols or triols (e.g., polyethylene glycol, glycerol, linear aliphatic diol (e.g., butanediol, hexanediol, octanediol, decanediol, dodecanediol, and shorter or longer linear aliphatic diols), etc.).

In some embodiments, a polymer is the polyesterification product of at least citric acid and one or more linear aliphatic diols (butanediol, hexanediol, octanediol, decanediol, dodecanediol, or any linear aliphatic diol from about 2-20 carbons in length). A polymer may comprise only citric acid and linear aliphatic diol components or may further comprise additional monomer components (e.g., sebacic acid, polyethylene glycol, glycerol, etc.). In some embodiments, a polymer comprises additional substituents or functional groups appended to the polymer (e.g., ascorbic acid, glycerol, a NONOate group, etc.).

In some embodiments, a polymeric component implant (e.g., stent) comprises citric acid as a monomer (e.g., along with a diol monomer). Citric acid is a reactive tricarboxylic acid that is part of the Krebs cycle and has been used as a key reactant monomer for the synthesis of polydiolcitrates with a wide range of properties and uses (Yang, J., et al., Synthesis and evaluation of poly(diol citrate) biodegradable elastomers. Biomaterials, 2006. 27(9): p. 1889-98; U.S. Pat. Nos. 8,772,437; 8,758,796; 8,580,912; 8,568,765; U.S. Pub. No. 2014/0155516; U.S. Pub. No. 2014/0135407; herein incorporated by reference in their entireties). Depending on the diol of choice, materials with controllable elasticity, biodegradability, and antioxidant properties can be developed (Serrano et al. Adv Mater, 2011. 23(19): p. 2211-5; Yang J., et al., A thermoresponsive biodegradable polymer with intrinsic antioxidant properties. Biomacromolecules, 2014. 15(11):3942-52; U.S. Pub. No. 2014/0037588; herein incorporated by reference in its entirety).

In some embodiments, an implant is 3D printed from selected materials to comprise the appropriate shape and dimensions. Systems, methods, and materials for manufacture of biomedical implants such as stents are described in, for example, U.S. Pub. No. 2021/0008246 and U.S. Pub. No. 2018/0117219, both of which are herein incorporated by reference in their entireties.

In some embodiments, implants (e.g., stents) contain therapeutics or other bioactive agents that can elute into the treatment location when implanted in a subject. In some embodiments, implants (e.g., stents) may comprise cells or other active agents for delivery to a subject. In some embodiments, implants (e.g., stents) may be coated with bioactive agents or materials to impart a desired functionality to the implant.

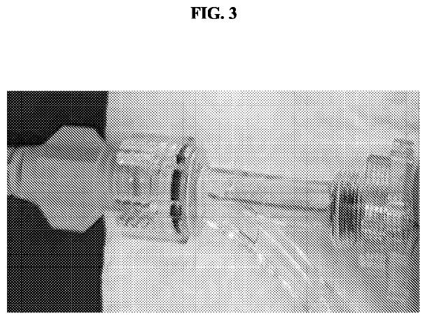

In some embodiments, the stent (or other implant) body comprises a tubular (cylindrical) shape with a length of 5-50 mm to (e.g., 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 55 mm, 50 mm, or ranges therebetween) and a diameter of 2-10 mm (e.g., 2 mm, 3 mm, 4 mm, 5 mm, 6 mm, 7 mm, 8 mm, 9 mm, 10 mm). In some embodiments, the length and diameter of the stent (or other implant) is selected based on the subject and location of placement. In some embodiments, an implant has a webbed structure (FIG. 3A). Shapes and features of stents, particular to their particular placement locations will be understood by those in the field. The sizes (e.g., lengths, widths, diameters, etc.) of the implant-placement systems herein (and the components thereof) are sized based on the placement location, type of implant, and the size of the implant to be placed.

In some embodiments, the implant is a coronary stent, a mesh-like, tubular device made of biocompatible materials.

In some embodiments, an implant deployment system herein comprises a guidewire, a balloon, a stopper, and a sheath. In some embodiments, a system further comprises an implant (e.g., stent (e.g., BVS), etc.) for placement within a subject. In some embodiments, a system further comprises a guiding catheter for insertion of the system into a subject. In some embodiments, the guiding catheter comprises a hemostatic valve at the proximal end of the guiding catheter to allow insertion of the system into the subject.

In some embodiments, systems herein comprise a guiding catheter. In some embodiments, the guiding catheter serves as the main access point to the treatment site (e.g., vasculature (e.g., arteries (e.g., coronary arteries)), digestive tract, biliary system, urethra, etc.).

In some embodiments, a guiding catheter provides a conduit for the insertion and advancement of other systems, devices, and/or components described herein. The catheter is made from flexible and biocompatible materials to ensure safe and smooth navigation through the vascular system. The guiding catheter is typically made of flexible, biocompatible materials such as polyurethane or polytetrafluoroethylene (PTFE). The guiding catheter comprises a lumen through which other devices and components of the systems herein are inserted and advanced to the treatment site. In some embodiments, at the proximal end of the guiding catheter, a hemostatic valve is incorporated to prevent blood backflow and maintain hemostasis during the procedure. This valve also facilitates easy insertion and removal of various instruments, devices, and/or components of the present system. In some embodiments, a guiding catheter comprises an outer shaft, inner lumen, and a hemostatic valve.

In some embodiments, the outer shaft of the guiding catheter is constructed from medical-grade polyurethane or polytetrafluoroethylene (PTFE). These materials offer the necessary flexibility for successful navigation to a treatment site (e.g., through the coronary anatomy). Other suitable materials will be understood by those in the field.

In some embodiments, inner lumen of the guiding catheter comprises and/or is coated with a low-friction material, such as a polymer, to facilitate easy advancement of the guidewire and other devices/components through the guiding catheter.

In some embodiments, the hemostatic valve is integrated at the proximal end of the guiding catheter. In some embodiments, the hemostatic valve is composed of silicone or similar elastomeric materials that allow easy insertion and removal of instruments while preventing blood backflow and maintaining hemostasis during the procedure. Other material for the hemostatic valve will be understood by the field.

In some embodiments, a guiding catheter comprises an outer diameter of the guiding catheter ranging from 3 French (F) to 10 F (1 F=0.33 mm), such as 3 F, 4 F, 5 F, 6 F, 7 F, 8 F, 9 F, 10 F, or ranges therebetween. In some embodiments, smaller diameters are used for less complex procedures, while larger diameters provide greater support for more challenging interventions. In some embodiments, guiding catheters come in various lengths, typically ranging from 20 cm to 150 cm (e.g., 20 cm, 30 cm, 40 cm, 50 cm, 75 cm, 100 cm, 125 cm, 150 cm, or ranges therebetween). The choice of length and diameter depends on the type of intervention, the location of the treatment site, the size/type of the implant, the patient's anatomy, and the operator's preference.

In some embodiments, a flexible guidewire is used to navigate through the subject (e.g., the coronary vasculature) to the treatment site, guiding the subsequent placement of the other components of the systems herein. In some embodiments, a guidewire is a flexible, thin wire-like device used to navigate through a subject to the treatment site (e.g., through the coronary arteries) and provides a pathway for the delivery of other devices/components to the target site. It is designed to be maneuverable and trackable through a subject (e.g., through the vascular anatomy).In some embodiments, a guidewire is coated with hydrophilic materials to reduce friction and improve maneuverability. In some embodiments, the distal end of a guidewire is shaped to facilitate smooth passage through the interior of a subject (e.g., through arterial bends and stenotic lesions). In some embodiments, the core wire of the guidewire is made from stainless steel, which provides excellent flexibility and torqueability. In some embodiments, the guidewire is coated with a hydrophilic material, such as polytetrafluoroethylene (PTFE) or hydrophilic polymer, to reduce friction and improve navigation through the subject. In some embodiments, the guidewire diameter is typically between 0.25 mm 0.60 mm and 150 cm to 300 cm, providing sufficient length for access to a treatment site through the guiding catheter.

In some embodiments, systems herein comprise a balloon catheter. In some embodiments, the balloon is used to hold the implant in place within systems herein during deployment, dilate treatment site, expand the implant (e.g., stent) for deployment, etc. In some embodiments, a balloon catheter comprises an inflatable balloon mounted on an elongate shaft. In some embodiments, the catheter is introduced over the guidewire and positioned across the treatment site (e.g., a narrowed segment of the coronary artery). Once in place, the balloon is inflated (e.g., with a radiopaque contrast medium, exerting radial force outward. The outward force of the balloon is used to hold the implant in place within systems herein during deployment, dilate treatment site (e.g., to widen vessel walls), expand the implant (e.g., stent) for deployment, etc. against the vessel walls.

In some embodiments, balloon catheters are provided in various sizes to accommodate different uses (e.g., treatment site diameters). The diameter of the balloon varies depending, for example, on the intended use and the size of the treatment site. Balloon diameters may range from 0.5 mm 10 mm (e.g., 0.4 mm, 1 mm, 2 mm, 3 mm, 4 mm, 5 mm, 6 mm, 7 mm, 8 mm, 9 mm, 10 mm, or ranges therebetween). In some embodiments, balloon lengths range from 5 mm to 50 mm (e.g., 5 mm, 10 mm, 15 mm, 20 mm, 30 mm, 40 mm, 50 mm, or ranges therebetween), depending, for example, on the intended use and the size of the treatment site. In some embodiments, the balloon catheter length, including the balloon and shaft, typically ranges from 75 cm to 200 cm (e.g., 75 cm, 100 cm, 125 cm, 150 cm, 175 cm, 200 cm, or ranges therebetween), depending, for example, on the intended use and the location of the treatment site.

The balloon is usually made from high-quality materials like nylon or polyethylene terephthalate (PET). These materials offer high tensile strength and excellent inflation characteristics. In some embodiments, the catheter shaft is made from a combination of materials, such as polyethylene, polyamide, or polyimide, to provide flexibility and pushability. Alternative materials are within the scope herein.

In some embodiments, systems herein comprise a sheath. In some embodiments, the sheath is a thin and/or flexible tubular component of sufficient length and diameter to encompass the implant (e.g., in a compressed or compacted conformation), the balloon (e.g., a deflated or partially-inflated state), the guidewire, the catheter shaft, and the stopper within a lumen of the sheath. In some embodiments, the sheath provides an outer protective covering for the devices/components of the systems herein being inserted into the body. In some embodiments, the sheath protects the components of the device from the interior environment of the treatment site and/or the path thereto prior to final deployment and expansion of the implant. In some embodiments, the sheath is made from a polytetrafluoroethylene (PTFE) or other medical grade materials, such as Polyether Ether Ketone (PEEK), Polyethylene (PE), Polyurethane (PU), or combinations thereof. In some embodiments, the sheath isa a tube-like structure with inner diameter ranging from 1 mm to 2 mm (e.g., 1.0 mm, 1.2 mm, 1.4 mm, 1.6 mm, 1.8 mm, 2.0 mm, or ranges therebetween), wall thickness of 0.01 to 0.1 mm (e.g., 0.01 mm, 0.02 mm, 0.05 mm, 0.1 mm, or ranges therebetween), and length ranging from 50 cm to 250 cm (e.g., 50 cm, 75 cm, 100 cm, 150 cm, 200 cm, 250 cm, or ranges therebetween) depending on the type of balloon catheter used.

In some embodiments, systems herein comprise a stopper. In some embodiments, the stopper is stably positioned on the guidewire and/or the shaft of the balloon catheter. In some embodiments, the stopper functions to prevent the implant from backsliding (e.g., traversing proximally along the catheter) when the system is inserted into a subject and/or moved through a subject. In some embodiments, the stopper functions to prevent the implant from backsliding (e.g., traversing proximally along the catheter) when the sheath is retracted from the treatment site (e.g., revealing the implant to the treatment site). In some embodiments, the stopper is of any suitable shape. In certain embodiments the stopper is cylindrical, but any shape that has one or more cross-sectional dimensions that prevent the implant (e.g., in the compressed or collapsed conformation) from sliding over the stopper. In some embodiments, the compressed or collapsed implant cannot pass over the stopped. In some embodiments, the stopper is not compressible. In some embodiments, the stopper has cross-sectional dimensions (e.g., width, height, diameter) that are less than the inner diameter of the lumen of the sheath (e.g., the largest cross-sectional dimension of the stopper is 95%, 90%, 85%, 80%, 75%, etc. of the diameter of the sheath).

In some embodiments, the stopper is made of any suitable material that is capable of resisting force applied by the implant in the proximal direction as a result of the retraction of the sheath. In some embodiments, the stopped comprises Polyetheretherketone (PEEK) or other medical grade materials, such as, polytetrafluoroethylene (PTFE) Polyethylene (PE), Polyurethane (PU), or combinations thereof. In some embodiments, the stopper is a tubular or cylindrical structure with an inner diameter of 0.5 mm to 1.5 mm (e.g., 0.5 mm, 0.75 mm, 0.85 mm, 1.0 mm, 1.25 mm, 1.5 mm, or ranges therebetween) and an outer diameter that is 0.05 to 0.5 mm (e.g., 0.05, 0.1 mm, 0.2 mm, 0.5 mm, or ranges therebetween) smaller than the sheath inner diameter. In some embodiments, the stopper is 2-8 mm in length (e.g., 2 mm, 3 mm, 4 mm, 5 mm, 6 mm, 7 mm, 8 mm, or ranges therebetween). In some embodiments, the stopper is immobilized (e.g., on the shaft of the catheter, for example, by an adhesive.

In some embodiments, systems herein may comprise various additional components or elements. For example, the implant (e.g., stent may be coated or impregnated with medications, cells, or other agents for deliver to the treatment site.

In some embodiments, systems herein comprise control for controlling various aspects of the system (e.g., steering, advancing, retracting components, inflating the balloon, etc.). In some embodiments, control is provided by an integrated hand-held control mechanism and/or handle mounted on the proximal end of the system (e.g., catheter). In some embodiments, the control mechanism/handle can be of various types, and adapted for operating a steerable catheter wherein the bend of the catheter can be selectively controlled by the operator. In some embodiments, controls are an integral part of the handle portion of the catheter. In some embodiments, controls and/or steering mechanisms are part of a separate unit attached to, or operable connected to a catheter. In some embodiments, the mechanism/handle includes a set of controls, which allow the operator to control the steering of the catheter and other operational functions of the catheter. It will be apparent to one of ordinary skill in the art that other control mechanisms/handles can be employed with the systems of the invention without departing from the scope thereof. Specifically, systems can include joystick controls for operating the steerable catheters and can include controls for rotating the angle at which the distal end of the catheter bends. Other modifications and additions can be made to the control mechanism/handle without departing from the scope of the invention. In some embodiments, the control mechanism/handle controls therapeutic-delivery functionalities, steering of the catheter, and any other functions that are understood by one in the art.

In some embodiments, systems herein comprise one or more imaging functionalities (e.g., intravascular imaging devices). These devices, such as ultrasound or optical coherence tomography (OCT), offer real-time visualization of the system and/or implant and aid in determining the appropriate placement.

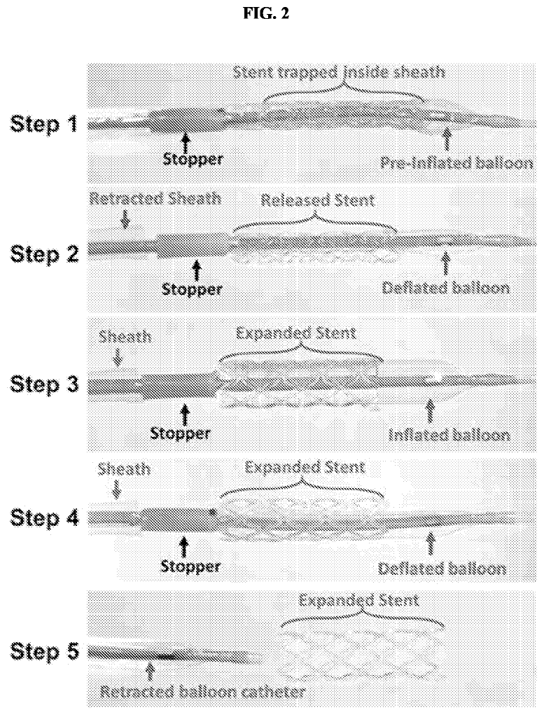

In some embodiments, provided herein are methods are for the placement of an implant at a treatment site within a subject using the systems and components therefore described herein. For example, the deployment of an exemplary BVS was achieved by the following steps (as shown in FIG. 2): Step 1, pre-inflate the balloon to 1.4 mm in order to ensure the firm contact between the balloon and BVS that are trapped inside the sheath and prevent the dislodgement between the BVS and balloon while advancing the device inside the vessel; Step 2, once the device reaches the targeted lesion, the balloon is deflated, and the sheath is retracted by pulling the proximal end of the sheath backward by at least 10 mm, i.e. the length of the BVS, in order to release the BVS (BVS would be moved backward with the sheath initially and stopped by the stopper at its distal end); Step 3, the balloon is inflated to the targeted diameter to fully expand the BVS; Step 4, the balloon is deflated while the BVS remains expanded; Step 5, the deployment system (including balloon catheter, stopper, and sheath) is retracted and the expanded BVS stays inside the targeted lesion.

The methods herein are not limited to the precise exemplary embodiment provided in FIG. 2.

In some embodiments, methods provide a step of placing a guiding catheter into an opening in the subject. The opening may be an incision or a natural orifice. In some embodiments, the guiding catheter provides an entry point into the subject for insertion of additional components of the systems herein. In some embodiments, the guiding catheter extends from the opening in the subject to the treatment site. In other embodiments, the guiding catheter only extends partially along the interior pathway between the opening and the treatment site.

Some embodiments herein utilize a guidewire to initially traverse the path between the opening in the subject at the treatment site. In such embodiments, a guidewire is inserted into the opening (incision or natural orifice) and guided along the path through the subject (e.g., through a blood vessel) to the treatment site. In some embodiments, the guidewire is insert through a guiding catheter. In some embodiments, once the distal end of the guidewire is at the treatment site, additional components can be inserted and placed as the treatment site along the guidewire.

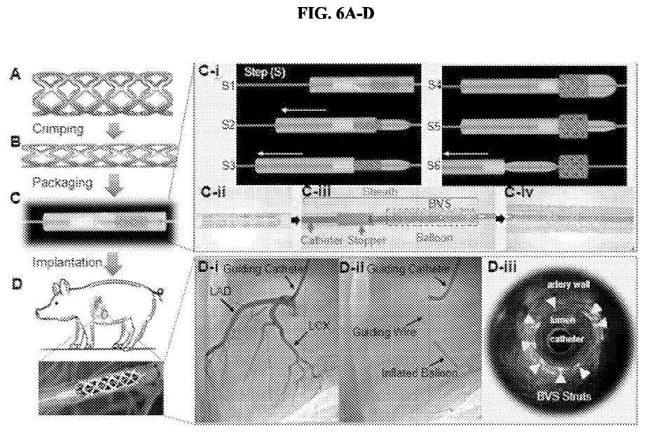

In some embodiments, the catheter, balloon, stopper, sheath, and implant are inserted into the subject and placed at the treatment site as a single assembled unit (e.g., as depicted in FIG. 1, Step 1 of FIG. 2, Step S1 of FIG. 6C-i, etc.).

In some embodiments, Heparin or other agents are applied to the system prior to insertion.

In some embodiments, the balloon is partially pre-inflated during or prior to insertion to produce a firm contact between the balloon and implant and/or sheath, thereby reducing/preventing undesired shift of the implant or sheath relative to the catheter and/or balloon during the advancement of system through the subject.

In some embodiments, the system is inserted into the guiding catheter in several steps. In some embodiments, a hemostatic valve of guiding catheter is opened while injecting saline to combat the backflow of blood from the hemostatic valve. In some embodiments, saline is injected by opening the valve partially (e.g., on a 3-way manifold). In some embodiments, the hemostatic valve is opened completely to decrease the friction of the balloon catheter with the hemostatic valve. In some embodiments, radiopaque markers on the balloon, catheter, or other components of the system are used to track the insertion and placement of the system in the subject. In some embodiments, the system is advanced toward the treatment site, making sure the sheath and catheter are advancing together without any relative shift.

In some embodiments, the system is advanced into the subject until the implant resides at the treatment site. In some embodiments, the balloon is deflated to release pressure from the balloon on the sheath and/or implant. In some embodiments, the sheath is then withdrawn/retracted to reveal the implant to the treatment site. If the implant is self-expanding or partially self-expanding, removing the sheath will result in full or partial expansion of the implant. The stopper prevents backsliding of the implant during retraction of the sheath. In some embodiments, the balloon is inflated to fully expand the implant. In some embodiments, following expansion of the implant, the balloon is deflated or partially deflated and the components of the system other than the implant are withdrawn from the treatment site.

Other steps in the placement of an implant (e.g., stent) with he systems described herein will be understood by those in the field.

### EXPERIMENTAL

### Example 1

### Fabrication of Bioresorbable Vascular Scaffold (BVS)

Methacrylated poly(1,12-dodecamethylene citrate) (mPDC) was synthesized as described previously (Van Lith, Robert, et al. Advanced Materials Technologies. 2016, 1.9: 1600138.). To formulate mPDC ink, 75 wt % mPDC was mixed with 2.2 wt % Irgacure 819 (MilliporeSigma) acting as a primary photoinitiator and 3.0 wt % Ethyl 4-dimethylamino benzoate (EDAB) (MilliporeSigma) being a co-photoinitiator, in a solvent of pure ethanol. A 3D printer based on micro-continuous liquid production process (μCLIP) was used to fabricate the BVS from prepared mPDC ink.

### Preparation of Balloon Delivery System (BDS)

A medical grade polyetheretherketone (PEEK) tube (Zeus Industrial Products, Inc, Orangeburg, NJ) with an outer diameter of 1.30 mm, inner diameter of 1.00 mm, and length of 5 mm was glued at the edge of tapered balloon on a catheter (Over-the-Wire) using liquid super glue (Loctite Corp., ONT), and serves as a stopper (FIG. 1). The BVS was crimped down to 1.0 mm using a crimping machine (RX550/650, Machine Solutions Inc., Flagstaff, AZ) and was put into a medical grade expanded polytetrafluoroethylene (e-PTFE) tube (Zeus Industrial Products, Inc.) with an inner diameter of 1.42 mm and wall thickness of 0.04 mm, which serves as a sheath to prevent the self-expansion of the BVS prior to the deployment. The sheath with a trapped BVS was put outside of the prepared balloon catheter with a stopper to finish the assembly of the BDS (FIG. 1).

### Example 2

### Exemplary BVS Design and Deployment

The fabricated BVS with and without drug were crimped and assembled on a customized delivery system (FIG. 6). Existing balloon catheter delivery systems do not work with the self-expanding BVS following the compression, which leads to the dislodgement between BVS and the balloon and the loss of the BVS. Existing balloon catheter systems were modified by introducing two components: the sheath and the stopper. The sheath, made by medical-grade expanded poly(tetrafluoroethylene), was used to lock the deformation of the compressed BVS following crimping (FIG. 6 C-ii). The locked BVS within the sheath was assembled on the balloon catheter with a stopper at the far end of the balloon (FIG. 6C and C-iii). During the deployment process (FIG. 6 C-i), the assembled device was first advanced into the targeted vessel (S1). While retracting the sheath, the BVS was first moved along with sheath till it was stopped by the stopper, which was made by a thin tube of medical-grade polyetheretherketone (S2), and then BVS would be released from the sheath (S3). The BVS was expanded against the surrounding vessel wall following the inflation (S4) and deflation (S5) of the balloon. Finally, the entire customized delivery system was retracted and withdrew from the body (S6). Through these steps, fabricated BVS with and without drug were successfully implanted into the domestic pig model (FIG. 6D). The angiographic images revealed the targeted arteries of left anterior descending coronary artery (LAD) and left circumflex artery (LCX) of a pig (FIG. 6D-i). A BVS was successfully expanded and deployed in the LCX of a pig (FIG. 6D-ii and D-iii).

### Example 3

### Exemplary BVS Deployment—Detailed Protocol

The guiding catheter (typically 7 F hockey stick, Amplatz Left 1.2, or Judkins Right) was inserted to reach near the coronary ostium.

The BVS balloon catheter was removed from the packaging and the BVS position was checked to make sure that the BVS was trapped inside the sheath (i.e., PTFE tube) and located on top of the balloon (FIG. 1, top). If the BVs is not properly positioned within the delivery system, BVS position is adjusted prior to further steps.

The sheath tube was Heparinzed using 10 U/mL sodium heparin (diluted 1000 U/mL to 10 U/mL using sterile saline). The syringe with 10 mL of 10 U/mL Heparin was placed over the stent end of the catheter and sheath. Gloved fingers were used to help seal the syringe to the sheath. The syringe plunger was depressed slowly; visually confirming that the sheath is filling with the heparin solution. The heparin solution does not have to fill the sheath completely. About one-third to one-half of the sheath should be filled. When the BVS system is placed into the guiding catheter, the subject's blood pressure will force some blood into the end of the system, pushing heparinized solution out towards open end. This heparinization procedure helps ensure that blood clots do not form on, or inside the sheath. Such clotting prevents smooth retraction of the sheath and can negatively affect patient mortality by acting as a nidus for intravascular thrombus formation.

The PTCA guidewire was inserted completely into the balloon catheter. Complete placement of the PTCA guidewire was confirmed by noting it's emergence from the tip of the balloon catheter and then pulling back into the balloon catheter.

The balloon was partially pre-inflated to make a firm contact between the balloon and stent/sheath (See FIG. 2, “Step 1”), which prevents the undesired relative shift between stent/sheath and balloon during the advancement of device. The balloon was inflated in 0.5 ATM increments with ˜10 seconds between each inflation for the delay in the balloon diameter. Care was taken not to overinflate, which can result in fracture of the sheath with ID 1.4 mm. The pre-inflated balloon should not bulge at the distal end. The system was hand tested to confirm that the stent/sheath cannot be easily shifted relative to the balloon.

The BVS/sheath/balloon catheter system was inserted into the guiding catheter in several steps. The hemostatic valve of guiding catheter was opened while injecting saline slowly to combat the backflow of blood from the hemostatic valve. Saline was injected by opening the valve partially on the 3-way manifold. Opening the hemostatic valve completely is essential to decrease the friction of the balloon catheter (and soft PTFE sheath) with the o-ring of the hemostatic valve. Loss of blood should not be a problem. The BVS device was inserted into the transparent section distal to the hemostatic valve to verify that the stent is in place within the sheath and the radiopaque markers of the balloon as shown in FIG. 3.

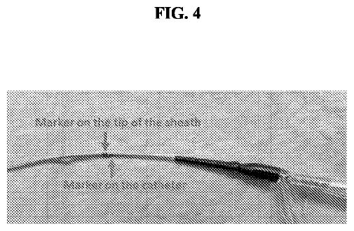

The BVS device was quickly advanced, making sure the sheath and catheter are advancing together without any relative shift by holding the sheath and catheter tightly (and having hemostatic valve wide open). Colored markers on the sheath and balloon catheter near the proximal end (outside the subject) of the balloon catheter remain aligned (FIG. 4).

The hemostatic valve was closed when fluoroscopy indicates that the tip of the guidewire or balloon markers were near the end of the guiding catheter, but not yet placed into the coronary artery.

The percutaneous transluminal coronary angioplasty (PTCA) guidewire was advanced in the target artery while holding the sheath/catheter still (by tightening the valve).

A catheterization assistant held the guidewire still, while the main catheterization operator opened the hemostatic valve and advanced the sheath/catheter together (without relative shift) down to the target artery.

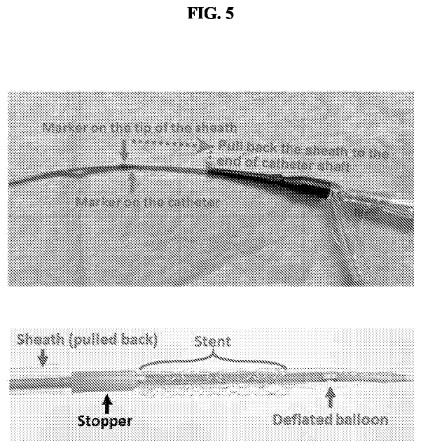

The valve was partially closed, the pre-inflated balloon was deflated, the catheter and guidewire were held still, and the sheath was retracted all the way to the end of the catheter (i.e. about 20 mm relative shift between sheath and balloon catheter (as shown by the dashed red line in FIG. 5).

Placement of the balloon at the desired artery site (e.g. atherosclerotic plaque, near arterial branch, etc.) was verified and the balloon was inflated to the desired size, thereby expanding the BVS within the treatment site (FIG. 2, “Step 3”).

The balloon was then completely deflated (FIG. 2, “Step 4”)., and after a 10 second delay, the deployment system was withdrawn leaving the expanded BVS in place (FIG. 2, “Step 5”).

Deployment of the BVS in the artery was verified with angiography and intravascular ultrasound (IVUS).

1. A catheter system for placement of an implant within a subject, the system comprising:
(a) a catheter comprising having distal and proximal ends;
(b) an implant for insertion within a subject, wherein the implant comprises a lumen, wherein the catheter extends longitudinally through the lumen of the implant, and wherein the implant is capable of adopting a compacted conformation and an expanded conformation;
(c) a stopper located at a position along the catheter that is proximal to the location of the implant on the catheter, wherein the stopper has a greater diameter than compacted conformation of the implant; and
(d) a sheath that extends longitudinally around the catheter and envelops the implant;
wherein the stopper prevents movement of the implant toward the proximal end of the catheter when the sheath is retracted toward the proximal end of the catheter.

2. The system of claim 1, wherein the catheter comprises a shaft and an inflatable balloon located at or near a distal end of the shaft.

3. The system of claim 2, all or a portion of the balloon resides within the lumen of the implant.

4. The system of claim 2, wherein the balloon is capable of adopting deflated and inflated states.

5. The system of claim 4, wherein the deflated balloon can reside within the implant in the compacted conformation, but the inflated balloon is too large to reside within the implant in the compacted conformation.

6. The system of claim 4, wherein a partially-inflated balloon can reside within the implant in the compacted conformation, but a fully-inflated inflated balloon is too large to reside within the implant in the compacted conformation.

7. The system of claim 1, wherein the catheter extends longitudinally through the stopper.

8. The system of claim 7, wherein the stopped is attached to the catheter and resides at a fixed position on the catheter.

9. The system of claim 2, wherein the sheath envelops the catheter, stopper, implant and balloon.

10. The system of claim 4, wherein a partially-inflated balloon exerts force on the inside of the sheath, thereby holding the implant and the sheath in place with respect to the catheter.

11. The system of claim 10, wherein sheath can retracted toward the proximal end of the catheter over the catheter, stopper, implant and deflated balloon.

12. The system of claim 11, wherein the sheath can be retracted to expose the stopper, implant and balloon.

13. The system of claim 1, wherein the implant is a stent.

14. The system of claim 13, wherein the stent is a coronary stent intended for placement in a coronary artery.

15. The system of claim 1, where the stent is a bioresorbable vascular scaffold (BVS).

16. The system of claim 1, wherein the implant is self-expanding or partially self-expanding.

17. The system of claim 16, wherein the sheath prevents the implant from adopting an expanded conformation by holding the sheath in the compacted conformation.

18. The system of claim 16, wherein the sheath prevents the implant from adopting an expanded conformation by preventing the sheath from contacting the physiologic environment outside the sheath

19. The system of claim 16, wherein retracting the sheath allows the implant to adopt the expanded conformation and/or to partially expand.

20. The system of claim 2, wherein retracting the sheath and inflating the balloon forces the implant to adopt the expanded conformation.

21. The system of claim 1, wherein the implant is a drug-eluting implant.

22. A method of placing an implant within a treatment site in a subject, the method comprising:
(a) inserting the system of one of claims 1-22 into a subject such that the implant is aligned with the treatment site;
(b) retracting the sheath toward the proximal end of the catheter, thereby exposing the implant to the treatment site; and
(c) allowing the implant to self-expand and/or applying mechanical outward force on the implant to place the implant into the expanded conformation within the treatment site.

23. A method of placing an implant within a treatment site in a subject, the method comprising:
(a) inserting the system of one of claims 2-22 into a subject such that the implant is aligned with the treatment site;
(b) retracting the sheath toward the proximal end of the catheter, thereby exposing the implant to the treatment site; and
(c) inflating the balloon to apply mechanical outward force on the implant to expand the implant into the expanded conformation within the treatment site.

24. The method of claim 23, wherein the balloon is in a non-inflated or partially-inflated state when the system is inserted into the subject and the implant is aligned with the treatment site.

25. The method of claim 24, further comprising a step after step (a) of deflating the balloon.

26. The method of claim 25, wherein the balloon is deflated before step (c).

27. The method of claim 26, wherein the balloon is deflated before step (b).

28. The method of claim 23, further comprising retracting the catheter from the treatment site.
